(19) United States
(12) Patent Application Publication
(10) Pub. No.: US 2024/0293441 A1
(43) Pub. Date: Sep. 5, 2024

(54) NEW THERAPEUTIC USE OF TYPE 2 IODOTHYRONINE DEIODINASE (D2) INHIBITORS

(71) Applicant: MATERIAS S.R.I, NAPOLI (IT)

(72) Inventors: Domenico SALVATORE, Napoli (IT); Maria Angela DE STEFANO, Casavatore (NA) (IT); Cristina LUONGO, Napoli (IT); Raffaele AMBROSIO, San Marzano Sul Sarno (SA) (IT); Tommaso PORCELLI, Roma (IT); Monica DENTICE, Napoli (IT)

(73) Assignee: CHEIRONTECH S.r.l., Napoli (IT)

(21) Appl. No.: 18/564,953

(22) PCT Filed: May 30, 2022
(86) PCT No.: PCT/EP2022/064534

§ 371 Date: Nov. 28, 2023

(30) Foreign Application Priority Data
Jun. 1, 2021 (IT) 102021000014333

(51) Int. Cl. A61K 31/714; A61K 31/12; A61K 31/192; A61K 31/198; A61K 31/343; A61K 31/352; A61K 31/4164; A61K 31/513; A61K 31/573; A61P 21/00

(52) U.S. Cl. CPC A61K 31/714; A61K 31/12; A61K 31/192; A61K 31/198; A61K 31/343; A61K 31/352; A61K 31/4164; A61K 31/513; A61K 31/573; A61P 21/00

(57) ABSTRACT

Inhibitor compounds of type 2 iodothyronine deiodinase (D2) are described for use in the therapeutic treatment of muscle wasting and/or in a muscle and/or skin regenerative therapeutic method. A pharmaceutical composition comprising a type 2 iodothyronine deiodinase (D2) inhibitor compound in a pharmaceutically acceptable vehicle, as well as in combination with optional excipients, diluents and adjuvants, is also described.

The present invention relates to the field of therapeutic treatments of muscle and skin diseases, particularly muscle wasting disorders and muscle or skin tissue injuries.

Skeletal muscle atrophy, also known as muscle wasting, is a debilitating condition that slowly develops during aging (sarcopenia) as well as after prolonged inactivity or fasting, or rapidly appears in a variety of pathologies such as cancer (cachexia), muscle denervation, chronic kidney disease, heart failure, chronic obstructive pulmonary disease, sepsis, diabetes, liver cirrhosis, cystic fibrosis or dystrophies. Dystrophies are a group of genetic diseases that lead to muscle weakening by limiting motor skills.

Muscle wasting diseases are characterized by a massive and progressive loss of muscle mass with consequent loss of strength and muscle function. In general, the loss of muscle mass results in a deterioration of the patient's physical conditions ranging from postural instability to impaired respiratory and cardiac capacity. For instance, approximately 80% of advanced cancer patients show a severe loss of muscle mass, a condition called cancer cachexia. In these cancer patients, loss of muscle mass is associated with a poorer prognosis and reduced response to therapy.

Since muscle function is essential for breathing, movements, chewing, and swallowing, reduced muscle mass and function is associated with a higher morbidity and mortality as well as reduced quality of life.

Even though the prevalence of people suffering from any kind of muscle weakness is high, particularly from cachexia and sarcopenia, there is no available specific treatment for muscle wasting and muscle remains an under-medicated organ. Up to date, there are no effective drugs available for improving muscle mass and function, which are proven to be able to interfere with the pathophysiological mechanism (i.e. systemic inflammation) underlying the loss of muscle strength in muscle wasting diseases.

Current therapies for cachexia-sarcopenia are based on three different classes of drugs: ghrelin receptor agonists (anamorelin); monoclonal antibodies directed against inflammatory mediators responsible for cachexia and muscle wasting (e.g. Abs versus IL-1, IL-6, TNF-alpha, myostatin); and selective non-steroidal androgen receptor modulators (SARMs, e.g. enobosarm). The above-mentioned drugs have been tested in phase 2 and phase 3 clinical trials (Becker C. et al., Lancet Diabetes Endocrinol. 2015 3(12):948-57 2015; Temel et al., Lancet Oncol. 2016; 17: 519-531), which all showed drug preventive effects on muscle loss and body mass gain. However, so far, no clinical data have shown the ability of tested drugs to increase muscle strength and improve physical-mechanical function of the muscle.

Besides cachexia and sarcopenia, also physical injuries may lead to loss of muscle mass. Muscle injuries are usually a result of external forces, such as muscle contusions and muscle lacerations. Large muscle injuries have a limited healing capacity, and the repair process usually results in the formation of scar tissue. The presence of an intramuscular scar alters the normal muscle contraction vector reducing strength and increasing fatigue.

Mechanical or chemical trauma may also damage the skin. Skin injuries most commonly involve burns, lacerations, fractures and crush injuries. Typically, such skin disorders can result in loss of the epidermis and often portions of the dermis and even subcutaneous fat. The treatment of these injuries can be complex and may have significant impact on the patient's function and aesthetics. Additionally, impaired skin healing processes may cause chronic wounds, which represent a large and growing disease burden.

As aforesaid, muscle and skin damages may cause functional impairment and severe disability as well as cosmetic deformities. Consequently, these injuries generate an ongoing reconstructive and regenerative challenge in clinical work in order to enable tissue regeneration and reduce the complications of transplantation.

To promote muscle and skin repair and regeneration, different strategies have been developed within the last century and especially during the last few decades, including surgical techniques, physical therapy, biomaterials, and muscular tissue engineering as well as cell therapy. However inherent limitations of the current approaches highlight the need for alternative strategies.

There is therefore a strong need of providing new effective therapeutic strategies aiming at delaying or preventing the progression of diseases involving muscle wasting, particularly age-related muscle degeneration as well as muscular atrophy conditions secondary to pathologies.

There is also a strong need of providing new pharmacological approaches aimed at ameliorating muscle and/or skin injuries by promoting tissue repair and tissue functional regeneration.

These and other needs are met by an inhibitor compound of type 2 iodothyronine deiodinase (D2) for the therapeutic use as defined in appended claim 1 and a pharmaceutical composition comprising said D2 inhibitor compound as defined in appended claim 7. Preferred embodiments of the invention are defined in the dependent claims.

The appended independent and dependent claims form an integral part of the present description.

Type 2 iodothyronine deiodinase (D2) is an enzyme that plays a key role in the maintenance of circulating and tissue levels of thyroid hormones. As known in the art, the intracellular thyroid hormone concentration is regulated by three seleno-membrane proteins, the deiodinase enzymes type 1, 2, and 3 (D1, D2, D3) (Gereben B. et al, Endocr Rev. 2008 29(7):898-938). In particular, D1 and D2 convert the pro-hormone thyroxine (T4) to the active hormone T3. Conversely, D3 converts T3 (triiodothyronine) to T2 (diiodothyronine) and T4 to rT3 (reverse triiodothyronine), both inactive thyroid hormone metabolites.

As will be explained in more details in the experimental section that follows, the present inventors have observed for the first time that type 2 deiodinase (D2) enzyme is expressed in mitotically quiescent stem cells of both muscle and skin and showed that this enzyme plays a critical role in maintaining the quiescence state of these cells.

As known in the art, stem cells are critical for the regeneration and homeostasis of adult tissues. Further experiments conducted by the inventors revealed that, upon muscle injury, genetic in vivo depletion of D2 improved and accelerated regeneration of muscle and skin tissues compared to wild-type animals, particularly in acute regenerative conditions such as cardiotoxin-induced muscle injury and wound healing.

By further investigating the role of type 2 deiodinase in muscle and skin, the present inventors have surprisingly found that, in animal models of muscular atrophy, the expression of this enzyme dramatically increases in the cachectic muscles. Moreover, in genetic mouse models of D2 depletion, the absence of type 2 deiodinase activity prevented skeletal muscles from experimentally induced atrophy.

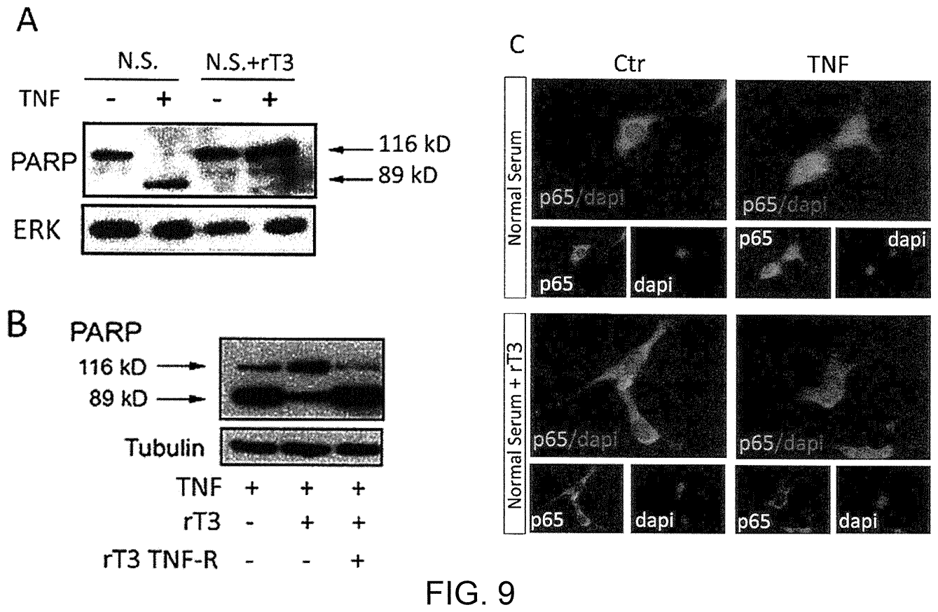

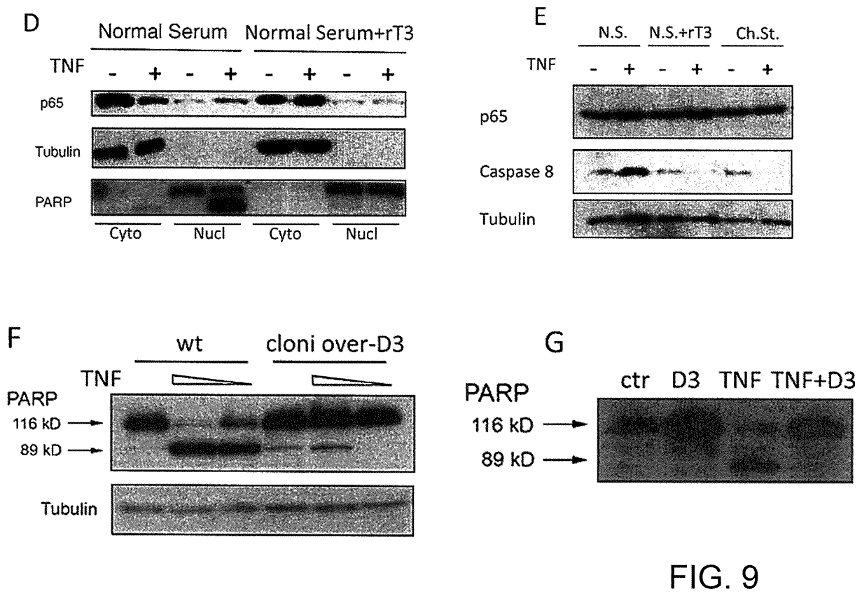

To further assess the effects of D2 modulation in muscle and skin as well as to exploit the role of this enzyme as a potential clinical target, the present inventors carried out dedicated pharmacological studies in vitro and in vivo by transiently blocking D2 activity using various inhibitor compounds. As shown in FIGS. 9, 10 and 11, consistent with the enclosed results obtained in pre-clinical models of atrophy, mice subjected to treatment with a D2 inhibitor compound were protected against denervation and cancer cachexia-induced muscle loss.

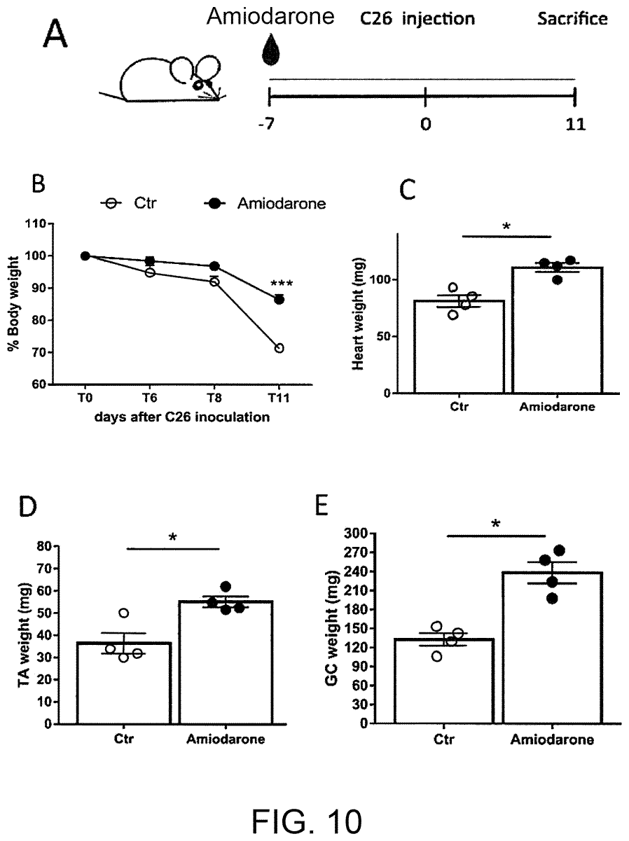

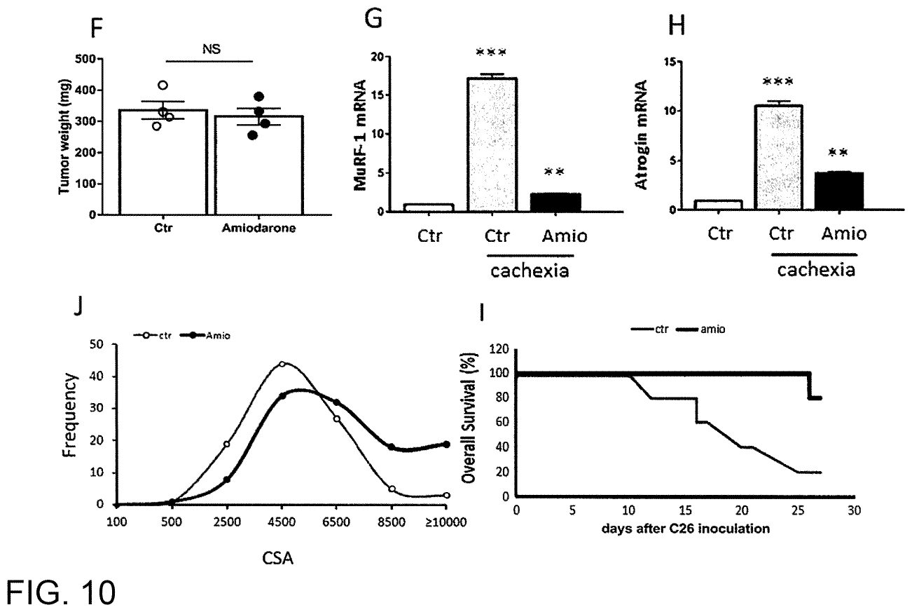

Notably, a significant increase in survival rate was also observed in tumor-bearing mice (FIG. 10I).

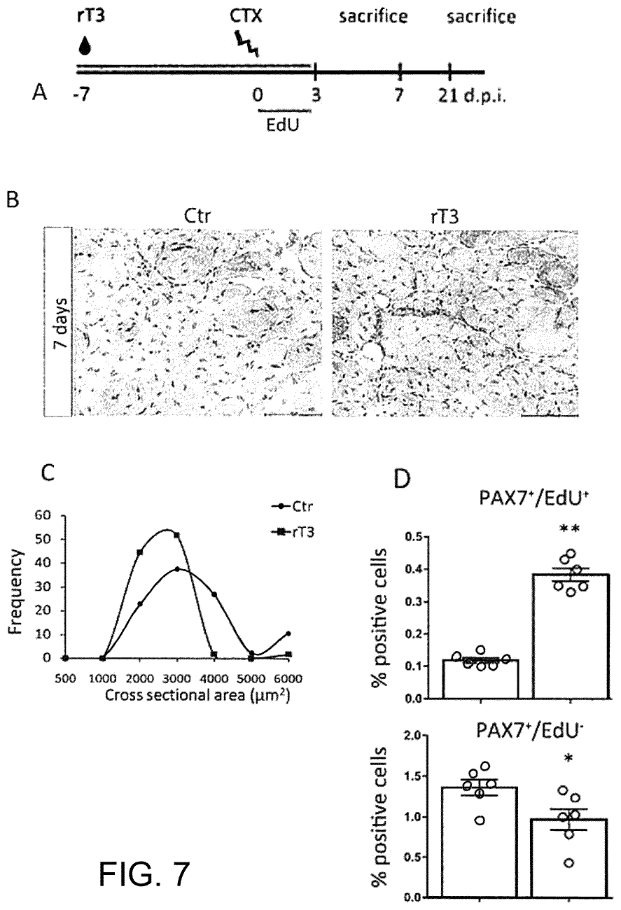

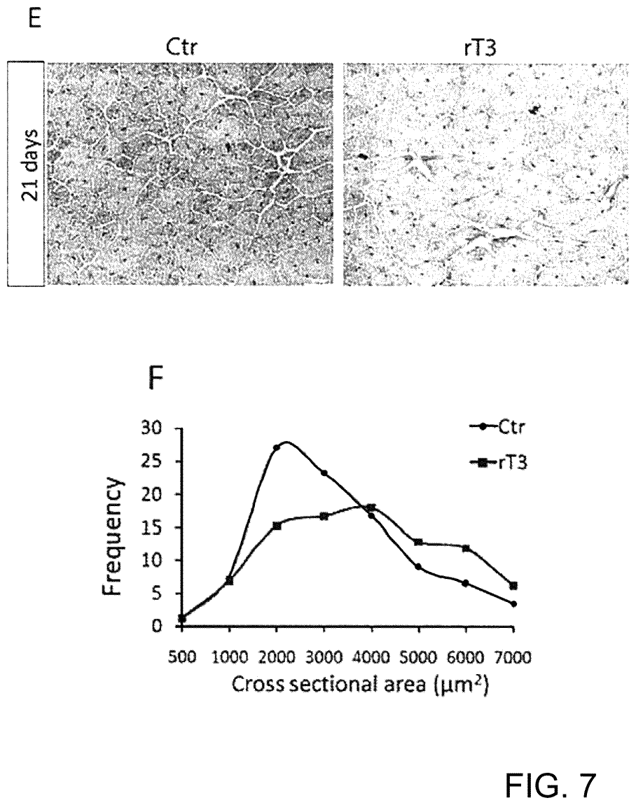

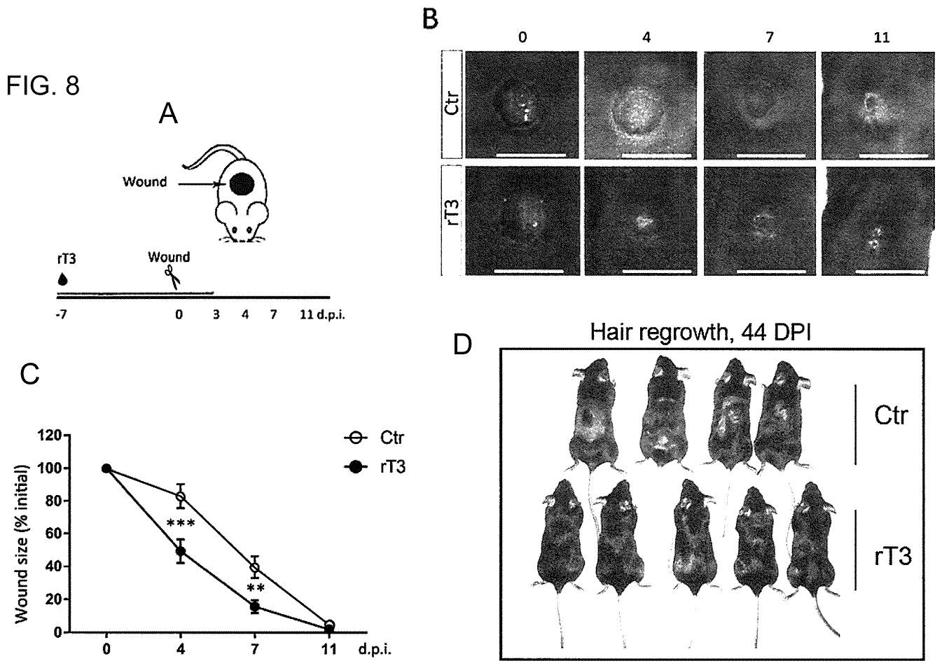

Additionally, in animal models of muscle or skin injury, the administration of a D2 inhibitor compound led to a marked increase in the number of muscle fibers and activated myoblasts in the damaged muscles of treated mice, compared to control animals (FIG. 7), as well as to a significantly more rapid and efficient wound healing process following skin injuries (FIG. 8).

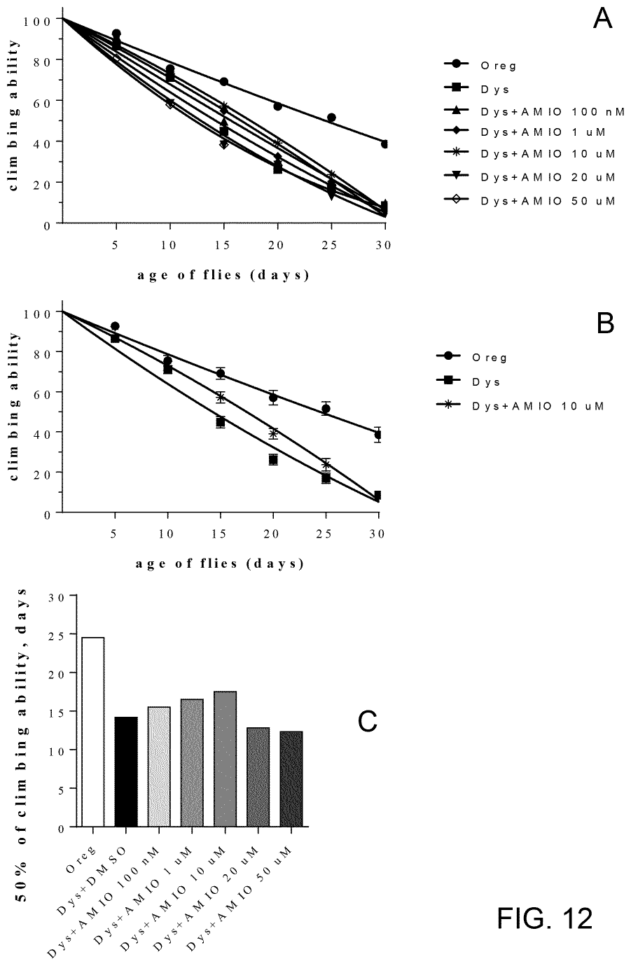

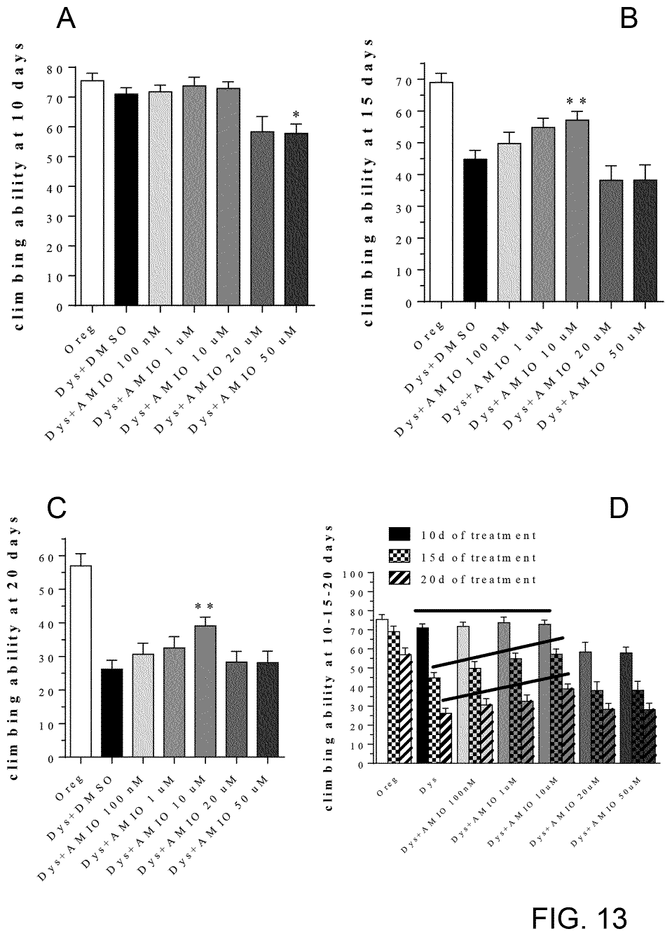

As further validation, the present inventors observed that in a Drosophila model of muscular dystrophy the chronic administration of flies with a D2 inhibitor compound enabled the functional recovery of skeletal muscle contractility (FIGS. 12 and 13).

Without wishing to be bound by any theory, the present inventors believe that the activation of thyroid hormone (TH) action in muscle tissue by the type 2 deiodinase enzyme is critical in triggering the accelerated muscle catabolismthat causes muscle loss in multiple disease states. The inventors further believe that D2 expression marks stem cells in a quiescent state in muscle and skin and that acute D2-depletion in quiescent stem cells triggers their spontaneous transition from G₀ into a G_{Alert} state, i.e. an intermediate state of the cell cycle between G₀ and G₁ phase, in which stem cells are more rapidly poised to enter the cell cycle in response to injury.

Based on all surprising findings as above illustrated, the attenuation of D2 enzymatic activity at tissue levels provides a novel and very effective therapeutic means to protect muscle against atrophy and/or to promote regeneration of damaged muscle and/or skin tissues.

A first aspect of the present invention is therefore an inhibitor compound of type 2 5 iodothyronine deiodinase (D2) for use in the therapeutic treatment of muscle wasting and/or in a muscle and/or skin regenerative therapeutic method.

As used herein, the term “inhibitor compound” refers to a substance capable of reducing and/or blocking the activity of an enzyme by binding to this protein and modifying the catalytic properties of the enzyme. Unless specified or apparent, the expression “inhibitor compound” is understood to encompass both selective and non-selective type 2 iodothyronine deiodinase inhibitors.

As used herein, unless otherwise specified, the term “therapeutic treatment” refers to the administration of the inhibitor compound of type 2 iodothyronine deiodinase (D2) according the invention after the onset of symptoms of the disease condition involving muscle wasting as well as to the administration prior to the onset of the symptoms, particularly to subjects at risk of the disease.

The term “regenerative therapeutic method”, as used herein, refers to therapeutic treatments aiming at re-growing, repairing or replacing damaged or diseased cells, tissues or organs in a subject.

In one embodiment, the inhibitor compound of type 2 iodothyronine deiodinase (D2) suitable to be used according to the invention is selected from the group consisting of reverse triiodothyronine (rT3), amiodarone (AMIO), desethylamiodarone (DEA), 5-methyl-2-thiouracil (MTU), 6-benzyl-2-thiouracil (BTU), xanthohumol (XTH), genistein (GEN), 6-Propyl-2-thiouracil (PTU), methimazole (MMI), iopanoic acid (IAc), dexamethasone, gold thioglucose (GTG), and any combination thereof.

Preferred D2 inhibitor compounds according to the invention are disclosed in Table 1 along with D2 susceptibility indices to inhibition by these compounds and related bibliographic references.

TABLE 1

| | Activity | References |
|---|---|---|
| Selective D2 inhibitors | | |
| Reverse | +++ | Nicod P. et al., J Clin Endocrinol |
| triiodothyronine (rT3) | | Metab. (1976) 43(2): 478-8; |
| | | Steinsapir J. et al., Endocrinology |
| | | (2000) 141(3): 1127-35 |
| Amiodarone (AMIO) | +++ | Rosene M. L. et al., Endocrinology |
| | | (2010) 151(12): 5961-70 |
| Desethylamiodarone | +++ | Rosene M. L. et al., Endocrinology |
| (DEA) | | (2010) 151(12): 5961-70 |
| Non selective D2 | | |
| inhibitors | | |
| 5-methyl-2-thiouracil | − | Rijnties E. et al., Eur Thyroid J. |
| (MTU) | | (2013) 2(4): 252-8 |
| 6-benzyl-2-thiouracil | −/+ | Rijnties E. et al., Eur Thyroid J. |
| (BTU) | | (2013) 2(4): 252-8 |
| Xanthohumol (XTH) | ++++ | Renko K. et al., Thyroid (2015) |
| | | 25(8): 962-8 |
| Genistein (GEN) | − | Renko K. et al., Thyroid (2015) |
| | | 25(8): 962-8 |
| 6-Propyl-2-thiouracil | + | Cettour-Rose P. et al., Eur J |
| (PTU) | | Endocrinol. (2005)153(3): 429-34; |
| | | Berry et al. Endocrine Reviews, |
| | | (1992) 13(2), 207-219. |
| | | Mandel et al. J Clin Endocrinol |
| | | Metab (1992) 75(4): 1133-1139. |
| Methimazole (MMI) | ++ | Rijnties E. et al., Eur Thyroid J. |
| | | (2013) 2(4): 252-8 |
| Iopanoic acid (IAc) | ++++ | Berry et al. Endocrine Reviews, |
| | | (1992) 13(2), 207-219. |
| | | Mandel et al. J Clin Endocrinol |
| | | Metab (1992) 75(4): 1133-1139. |
| Dexamethasone | ++ | Martinez-deMena R. et al., Mol Cell |
| | | Endocrinol. (2016)15; 428: 58-67 |
| Gold thioglucose | ++ | St. Germain D L. et al., |
| (GTG) | | Endocrinology. (2009) 150(3): 1097- |
| | | 107 |

Preferably, the inhibitor compound of type 2 iodothyronine deiodinase (D2) for use according to the invention is selected from reverse triiodothyronine (rT3), amiodarone (AMIO) and desethylamiodarone (DEA), more preferably from reverse triiodothyronine (rT3) and amiodarone (AMIO).

The reverse triiodothyronine compound (rT3, 3,3′5′-triiodothyronine, CAS number: 5817-39-0) is a metabolically inactive form of thyroid hormone, which is generated from the thyroxine (T4) pro-hormone by removal of an iodine atom in the inner ring of T4 via the type 3 5′-deiodinase enzyme.

Amiodarone (AMIO) is an iodinated benzofuran derivative primarily known for its approved indication in the treatment of cardiac diseases. This drug is a potent class III antiarrhythmic agent capable of inducing prolongation of the action potentials and refractory periods in the heart.

Desethylamiodarone (DEA) represents the major bioactive metabolite of amiodarone, and is produced in an N-demethylation reaction catalyzed by cytochrome P450 3A4.

A preferred embodiment according to the invention is reverse triiodothyronine (rT3) for use in the therapeutic treatment of muscle wasting due to a disease condition such as e.g. cachexia, sarcopenia or muscle denervation.

Another preferred embodiment according to the invention is reverse triiodothyronine (rT3) for use in a muscle and/or skin regenerative therapeutic method.

A still another preferred embodiment according to the invention is amiodarone (AMIO) for use in the therapeutic treatment of muscle wasting due to a disease condition such as e.g. cachexia, sarcopenia or muscle denervation.

A yet another preferred embodiment according to the invention is amiodarone (AMIO) for use in a muscle and/or skin regenerative therapeutic method.

According to the invention, it is envisaged that any possible combination of inhibitor compounds of type 2 iodothyronine deiodinase (D2) as above defined is encompassed within the present invention.

For example, in one embodiment of the invention rT3 is used in combination with 6-Propyl-2-thiouracil (PTU). Advantageously, in this embodiment, PTU blocks hepatic D1 activity in liver thus enhancing rT3 half-life.

As aforesaid, a therapeutic approach based on the administration of a D2 inhibitor compound is particularly suitable for the treatment of disease conditions involving loss of muscle mass and muscle strength such as, for example, secondary wasting pathologies (cachexia) or age-associated muscle degeneration (sarcopenia).

Besides protection against muscular atrophy, pharmacologically blocking type 2 deiodinase activity results in the stimulation of muscle and skin stem cells proliferation, thereby making tissue-specific modulation of the D2 enzyme an effective and innovative tool in therapeutic contexts for regenerative medicine.

By way of example, but without limitation, muscle wasting according to the invention may be due to a disease condition selected from the group consisting of sarcopenia, cancer, sepsis, diabetes, chronic heart failure, chronic obstructive pulmonary disease, chronic renal failure, liver cirrhosis, cystic fibrosis, muscle denervation, fasting, and any combination thereof.

A preferred embodiment of the present invention relates to a therapeutic method aiming at controlling neoplastic cachexia.

With reference to a muscle regenerative therapeutic method, exemplary diseases that can be treated according to the invention include, but are not limited to, delayed-onset muscle soreness (DOMS), muscular contusion, muscular strain, muscular laceration, cancer cachexia, muscle atrophy, muscle dystrophy, and any combination thereof.

In the context of skin regenerative therapeutic method, mention can be made, for example, but without limitation, of therapeutic treatment of ulcers, dermatitis, skin wounds, burns, lacerations, and any combination thereof.

The type 2 iodothyronine deiodinase (D2) inhibitor compound for use according to the invention may also be effectively administered in the form of a pharmaceutical composition.

Accordingly, a second aspect of the present invention is a pharmaceutical composition for use in the therapeutic treatment of muscle wasting and/or in a muscle and/or skin regenerative therapeutic method, said pharmaceutical composition comprising a therapeutically effective amount of at least one inhibitor compound of type 2 iodothyronine deiodinase (D2) as above defined and a pharmaceutically acceptable vehicle, excipient and/or diluent.

As used herein, the term “therapeutically effective amount” refers to an amount of the D2 inhibitor compound for use according to the invention sufficient to exhibit a detectable therapeutic effect. The precise effective amount for a subject will depend upon the subject's size and health, the nature and severity of the condition to be treated.

The term “pharmaceutically acceptable” refers to compounds and compositions which may be administered to mammals without undue toxicity at concentrations consistent with effective activity of the active ingredient.

The selection of vehicles, excipients and/or diluents suitable for the pharmaceutical composition of the invention can be determined by a person of ordinary skill in the art by using his/her normal knowledge.

Preferably, the at least one inhibitor compound of type 2 iodothyronine deiodinase in the pharmaceutical composition for use according to the invention is selected from reverse triiodothyronine (rT3), amiodarone (AMIO), desethylamiodarone (DEA), 5-methyl-2-thiouracil (MTU), 6-benzyl-2-thiouracil (BTU), xanthohumol (XTH), genistein (GEN), 6-Propyl-2-thiouracil (PTU), methimazole (MMI), iopanoic acid (IAc), dexamethasone, gold thioglucose (GTG), and any combination thereof.

A preferred pharmaceutical composition according to the invention comprises reverse triiodothyronine (rT3) and is suitable for use in the therapeutic treatment of muscle wasting due to a disease condition such as e.g. cachexia or sarcopenia.

Another preferred pharmaceutical composition according to the invention comprises reverse triiodothyronine (rT3) and is suitable for use in a muscle and/or skin regenerative therapeutic method.

A still another preferred pharmaceutical composition according to the invention comprises amiodarone (AMIO) and is suitable for use in the therapeutic treatment of muscle wasting due to a disease condition such as e.g. cachexia and sarcopenia.

A yet another preferred pharmaceutical composition according to the invention comprises amiodarone (AMIO) and is suitable for use in a muscle and/or skin regenerative therapeutic method.

It is also envisioned by the present invention that any combination of inhibitor compounds of type 2 iodothyronine deiodinase (D2) as above mentioned may be present in the pharmaceutical composition of the invention.

In one embodiment, the pharmaceutical composition for use according to the invention comprises rT3 in combination with any of amiodarone (AMIO), desethylamiodarone (DEA), 6-Propyl-2-thiouracil (PTU), and Iopanoic acid (IAc).

In another embodiment, the pharmaceutical composition for use according to the invention comprises amiodarone in combination with any of rT3, desethylamiodarone (DEA), 6-Propyl-2-thiouracil (PTU), and Iopanoic acid (IAc).

The pharmaceutical composition for use according to the invention is suitable to be administered as therapeutic treatment against muscle-related and/or skin-related diseases, and/or for repairing muscle and/or skin tissue injuries, all as defined above with reference to the therapeutic use of the D2 inhibitor compound.

The pharmaceutical composition for use according to the invention can be formulated into any suitable dosage form, for example for administration via the topical, oral, enteral or parenteral route.

As used herein, parenteral administration includes, but is not limited to, administration of a pharmaceutical composition by subcutaneous and intramuscular injection, implantation of sustained release depots, intravenous injection administration.

The pharmaceutical composition for use according to the invention may be administered by topical application in a solution, cream, ointment, spray, gel or any local application. The pharmaceutical composition of the invention may also be administered transdermally by means of a drug eluting device, such as gauze, a patch, pad, or a sponge.

The pharmaceutical composition of the invention may also be formulated in dosage forms for oral administration, such as a tablet, capsule, powder, granule, solution, suspension, syrup and the like.

In a pharmaceutical composition, the amount of the administered active ingredient can vary widely according to considerations such as the particular compound and dosage unit employed, the mode and time of administration, the period of treatment, the age, sex, and general condition of the subject treated, the nature and extent of the condition treated, the rate of drug metabolism and excretion, the potential drug combinations and drug-drug interactions.

The pharmaceutical composition for use according to the invention is administered as a single dose or as multiple doses, and as frequently as necessary and for as long of a time as necessary in order to achieve the desired therapeutic effect. One of ordinary skill in the art can readily determine a suitable course of treatment utilizing the pharmaceutical compositions for use according to the invention.

According to the embodiment where the pharmaceutical composition comprises reverse T3, the therapeutic regimen is preferably based on administering to the patient a daily dose of rT3 comprised between 50 micrograms (μg) and 500 μg.

According to another embodiment where the pharmaceutical composition comprises amiodarone, the therapeutic regimen is preferably based on administering to the patient a daily dose of amiodarone comprised between 100 mg and 700 mg, more preferably a daily dose of amiodarone comprised between 200 mg and 600 mg.

A preferred therapeutic regimen according to this embodiment comprises orally administering to the patient a regimen consisting of a daily dosage of 600 mg of amiodarone during the first two weeks of treatment, a daily dosage of 400 mg of amiodarone during the third week of treatment and a daily dosage of 200 mg of amiodarone during the maintenance period of therapy.

As known in the art, amiodarone therapy can potentially result in a wide range of adverse effects. Advantageously, said treatment regimen reduces the incidence of drug-related adverse effects without affecting amiodarone efficacy.

The following experimental section is provided purely by way of illustration and is not intended to limit the scope of the invention as defined in the appended claims. In the following experimental section reference is made to the appended drawings, wherein:

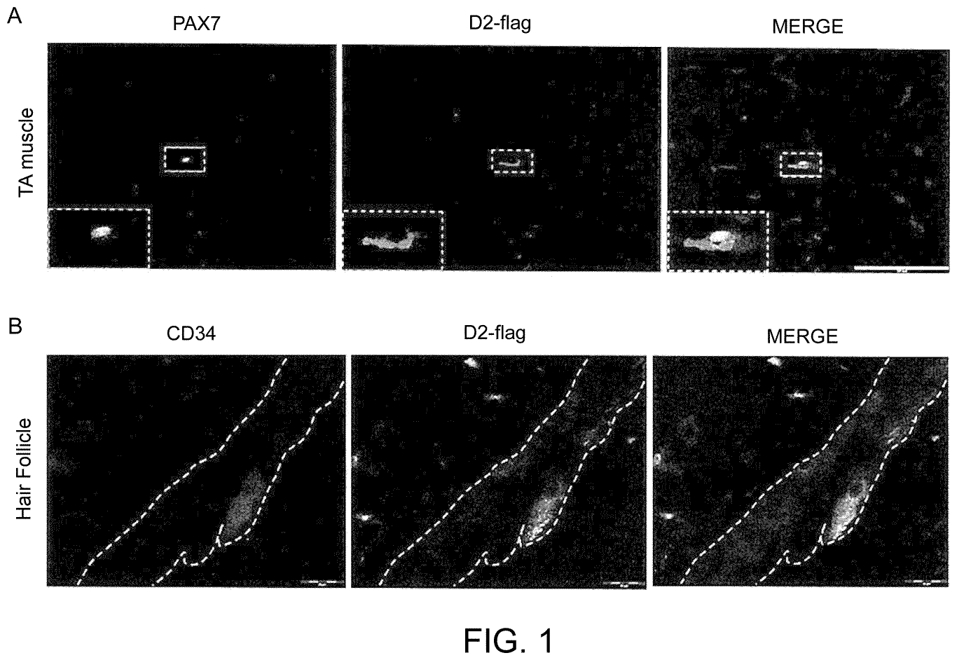

FIG. 1 shows that type 2 iodothyronine deiodinase (D2) is highly expressed in quiescent stem cells of different tissues. The results are based IF on Pax7-nGFP and D2-3xflag mice in muscle and skin stem cells (SCs). (A) Representative immunofluorescence (IF) staining of Pax7 (green) and D2-flag (red) expression on cryosections of uninjured tibialis anterior (TA) muscle (Scale bar, 50 μm). (B) Representative IF staining of CD34 (red) and Flag-D2 (green) expression in the hair follicle of the epidermis (scale bar);

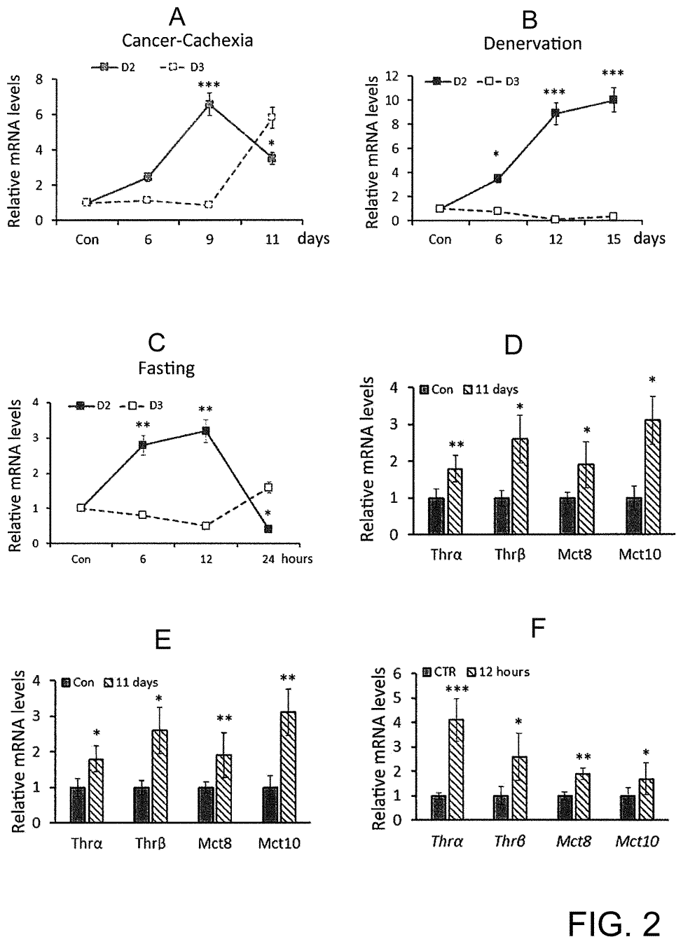

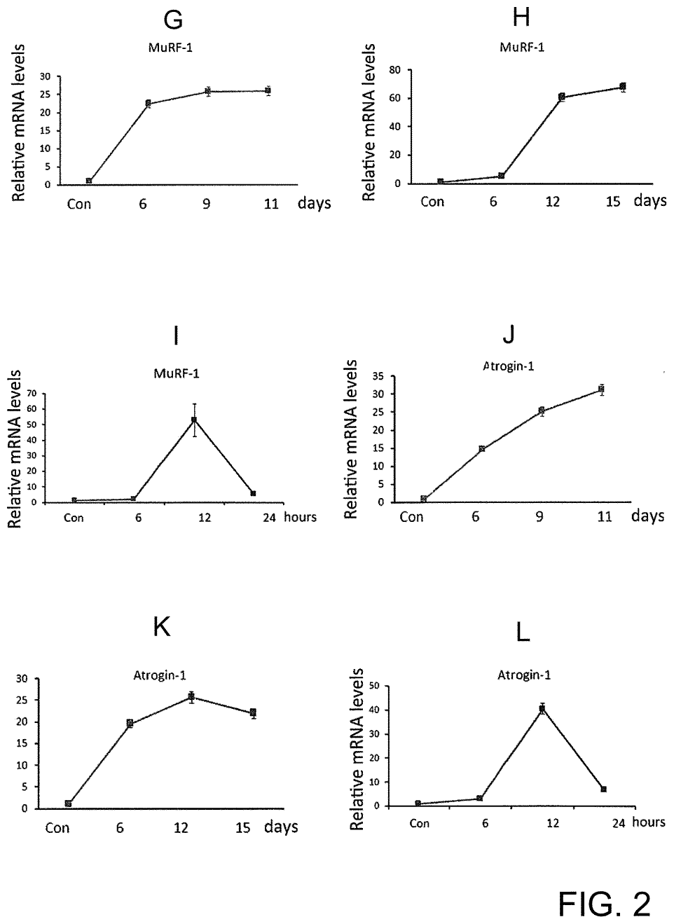

FIG. 2 shows that D2 expression in muscle is induced in different animal models of muscle wasting. D2 and D3 mRNA expression was determined in TA muscle (A) from cancer cachexia (C26-treated) animals sacrificed 6, 9, and 11 days after cancer cachexia induction, (B) from denervated mice sacrificed 6, 12, and 15 days after the denervation, and (C) from mice fasted for 6, 12, and 24 hours. TRα, TRβ (thyroid hormone receptors), MCT8 and MCT10 (thyroid hormone transporters) mRNA expression in TA muscle from cachexia (D), denervation (E) and fasting mice (F). MuRF-1 mRNA expression in TA muscle from cachexia (G), denervation (H) and fasting (I) mice. Atrogin-1 mRNA expression in TA muscle from cachexia (J), denervation (K) and fasting (L) mice;

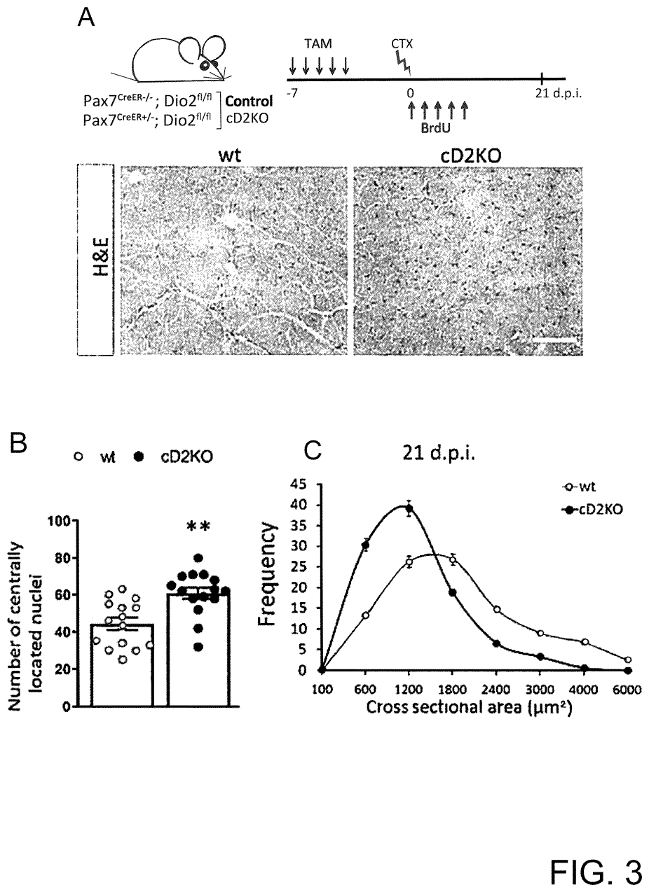

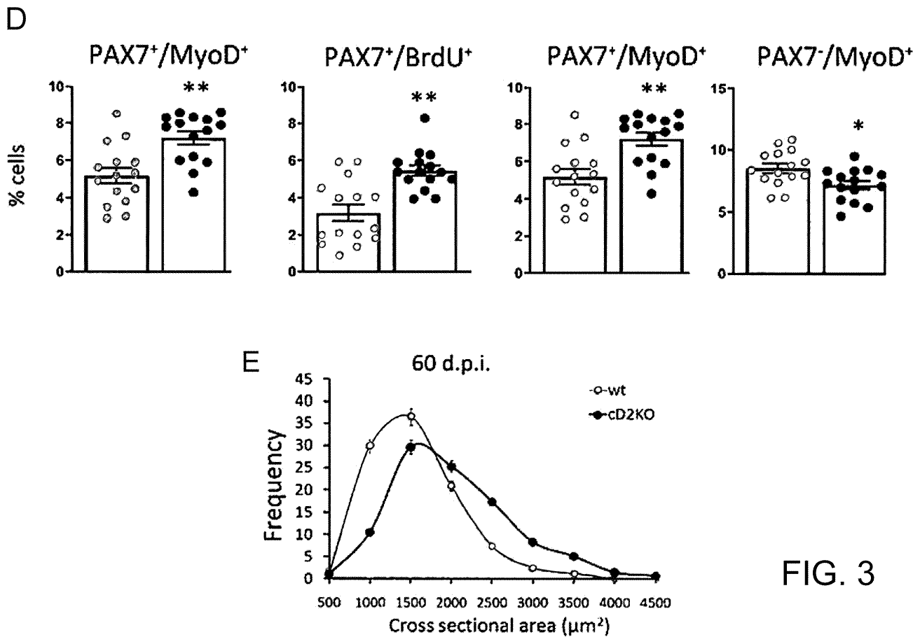

FIG. 3 shows that D2 depletion in quiescent stem cells accelerates muscle regeneration. (A) (top) Schematic diagram of the experimental design and (bottom) representative micrographs showing Hematoxylin and Eosin (H&E) staining of the TA sections from wild-type (wt) and deiodinase-deficient (cD2KO) mice after tamoxifen-induction (Scale bar, 100 μm). (B) Percentage of centrally located nuclei evaluated in the sections shown in A. (C) Quantification of the cross-sectional area of TA sections as shown in A. (D) Quantification of percentage positive cells by double staining Pax7/MyoD and Pax 7/BrdU (wt n=5; cD2KO n=5 in three individual experiments). (E) Quantification of the cross-sectional area analysis of TA sections from cD2KO and wt (Pax 7^{creER}−(Td/GFP) adult mice 60 days after CTX-injection;

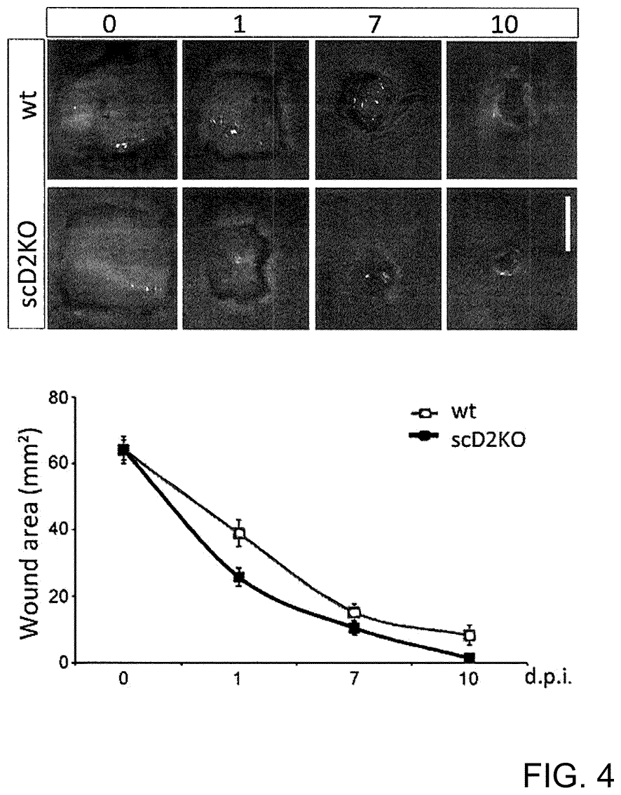

FIG. 4 shows that D2 depletion in quiescent stem cells accelerates skin regeneration. Wound healing experiment were performed in scD2KO mice and wt mice following tamoxifen-induced D2 depletion. (Upper panel) Representative images of the wound arca at 0, 1, 7 and 10 days following the wound healing. (Lower panel) Graph showing wound area calculated by using the Cell*F Olympus Imaging Software (Scale bar represents 1 cm).

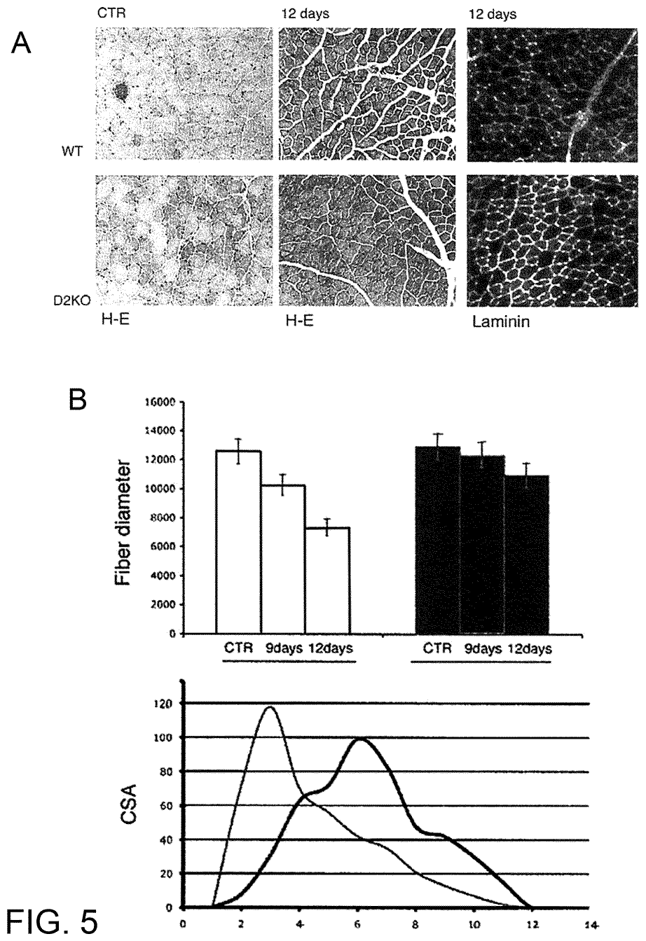

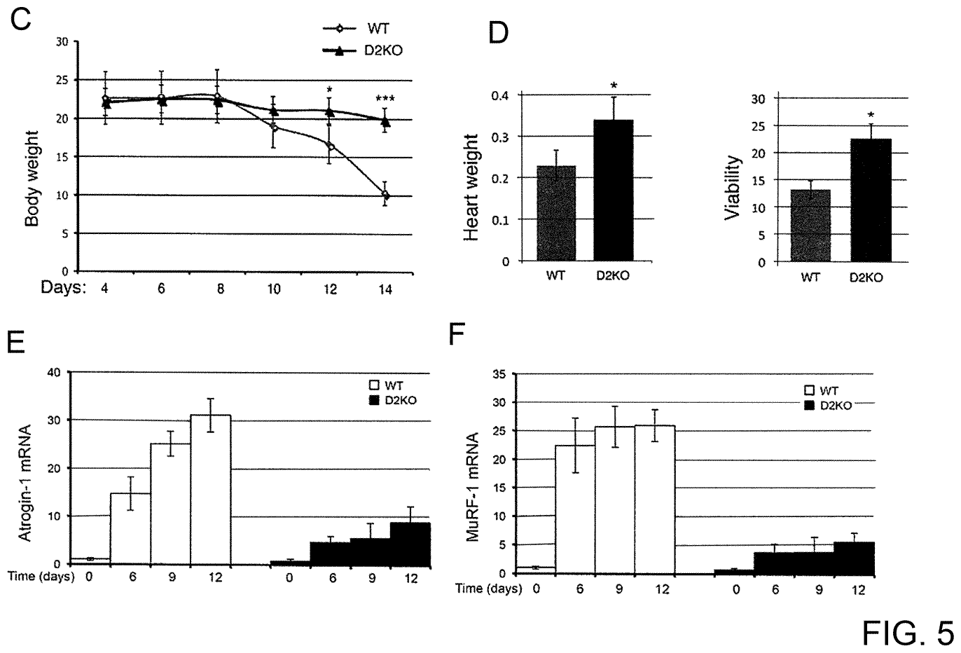

FIG. 5 shows that global D2 depletion in mice attenuates muscle wasting induced by cancer (cachexia). (A) Representative micrographs showing H&E staining and (B) graphs showing mean fiber diameter and frequency distribution of myofiber cross sectional arca (CSA) of TA muscles from wt and D2KO mice sacrificed 12 days after C26 cell injection. (C) Graph showing time course of body weight changes of wt and D2KO mice monitored during 14 days following the C26 cells injection. (D) Graphs showing heart weight and viability of wt and D2KO mice sacrificed 15 days after C26 cells injection. (E) Atrogin-1 and (F) Murf-1 mRNA expression in TA muscle from animals sacrificed 6, 9, and 12 days after C26 cells injection;

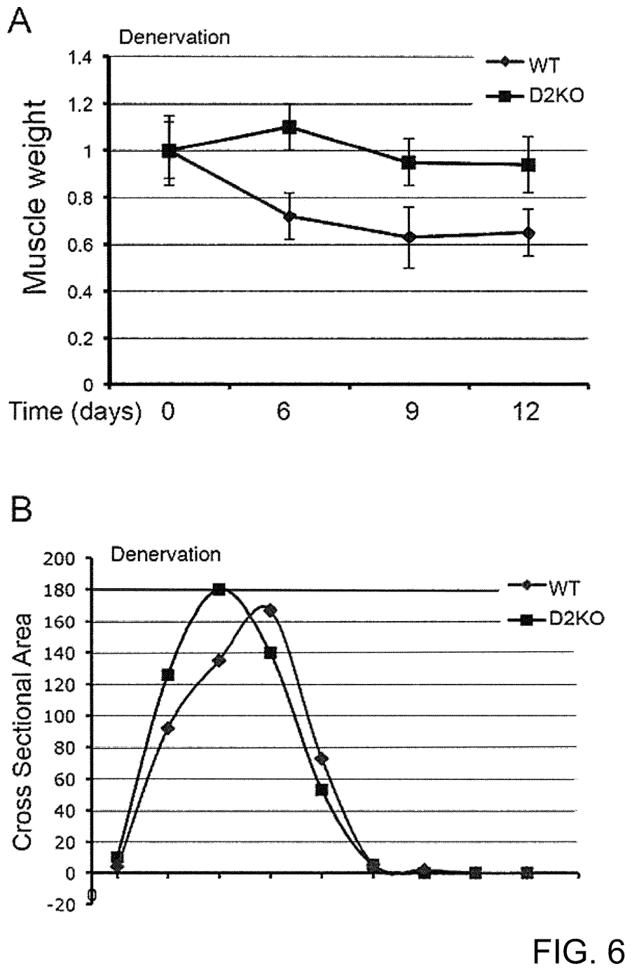

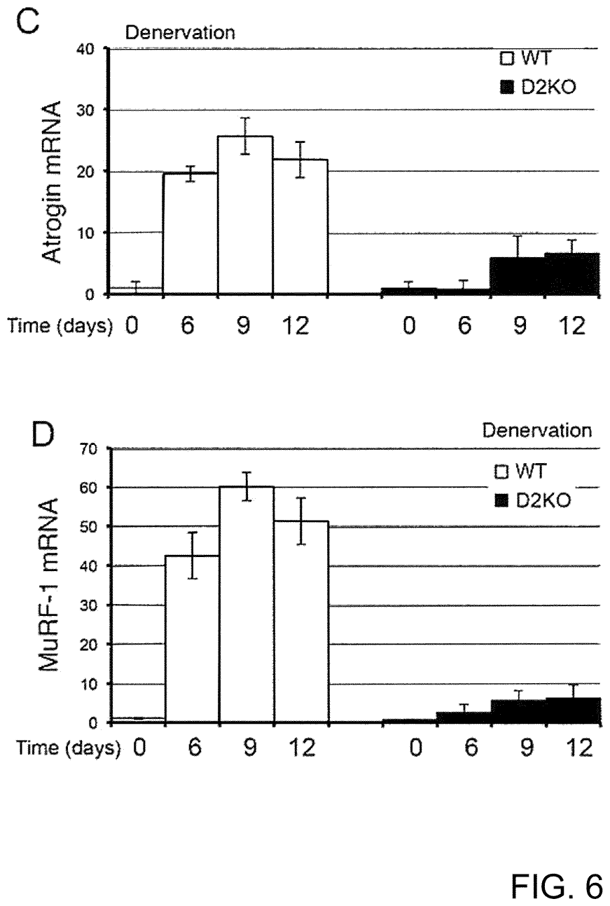

FIG. 6 illustrates that global D2 depletion in mice attenuates muscle wasting induced by denervation. (A) Graph showing time course of body weight changes of wt and D2KO mice monitored during 12 days following the hind limb denervation. (B) Graph representing frequency distribution of myofiber CSA of TA muscles from wt and D2KO mice sacrificed 12 days after denervation. (C) Atrogin-1 and (D) Murf-1 mRNA expression in TA muscle from animals sacrificed 6, 9, and 12 days after denervation;

FIG. 7 shows the therapeutic ability of rT3 to activate stem cells during muscle regeneration by blocking D2. (A) Schematic overview of the experimental design. (B) Representative micrographs showing H&E staining of TA sections from rT3-treated and untreated (control, ctr) mice, sacrificed 7 days after injury (Scale bar, 50 μm). (C) Quantification of the cross-sectional area of the tissue sections shown in B. (D) Graph showing the percentage of Pax7/EdU staining on TA sections from rT3-treated and ctr mice (Ctr=3; rT3=3 mice in two individual experiments). (E) Representative micrographs showing H&E staining of rT3-treated and Ctr mice, sacrificed 21 days after injury (Scale bar, 50 μm). (F) Quantification of the cross-sectional area of the tissue sections shown in E;

FIG. 8 shows the therapeutic ability of rT3 to activate stem cells during skin regeneration by blocking D2. (A) Schematic overview of the experimental design, (B) representative images of skin wound healing in mice treated with rT3 versus control, and (C) graph illustrating the quantification of wound closure presented as the mean of the percent of initial wound size (n=4 each group in four independent experiments) (Scale bar 1 cm). (D) Representative images of animals at 28 days post-injury. Animals were administered rT3 5 days before shaving, and skin wounding display improvements in hair regrowth. Data are represented as average±SEM; * p<0.05, ** p<0.01, *** p<0.001 using a Mann-Whitney test when comparing two conditions, and multiple T-test;

FIG. 9 shows that blocking D2 activity with rT3 attenuates muscular atrophy in vitro. (A) Representative image of immunoblot of PARP in pp6 cells. ERK serves as loading control. (B) Representative image of immunoblot of PARP measured in pp6 cells. Tubulin serves as loading control. (C) Representative IF staining of p65/NFKB on pp6 cells treated with or without rT3. (D) Representative image of immunoblot of p65/NFKB in pp6 cells. Tubulin and PARP serve as loading controls for cytosolic and nuclear compartments, respectively. (E) Representative image of immunoblot of p65/NFKB and caspase8 in pp6 cells. Tubulin serves as loading control. (F) Representative image of immunoblot of PARP in C2C12 cells. Tubulin serves as loading control. (G) Representative image of immunoblot of PARP in pp6 cells;

FIG. 10 shows that amiodarone treatment protects against muscle wasting in cancer cachexia mice models. (A) Schematic overview of the experimental design. (B) Time course of body weight changes of Ctr and Amiodarone-treated mice monitored over 11 days following the C26 cells injection. (C-E) Graphs showing (C) heart weight, (D) TA and (E) gastrocnemius muscle (GC) weight of Ctr and amiodarone-treated mice sacrificed 11 days after C26 cells injection. (F) Graph showing tumor weight in ctr and amiodarone-treated mice sacrificed 11 days after C26 cells injection. (G) MURF-1 and (H) Atrogin-1 mRNA expression in TA muscle from animals sacrificed 11 days after C26 cells injection. (I) Graph showing the overall survival (%) of Ctr and amiodarone-treated mice monitored following the C26 cells injection. (J) Frequency distribution of myofiber CSA of TA muscles from Ctr and amiodarone-treated mice sacrificed 11 days after cachexia;

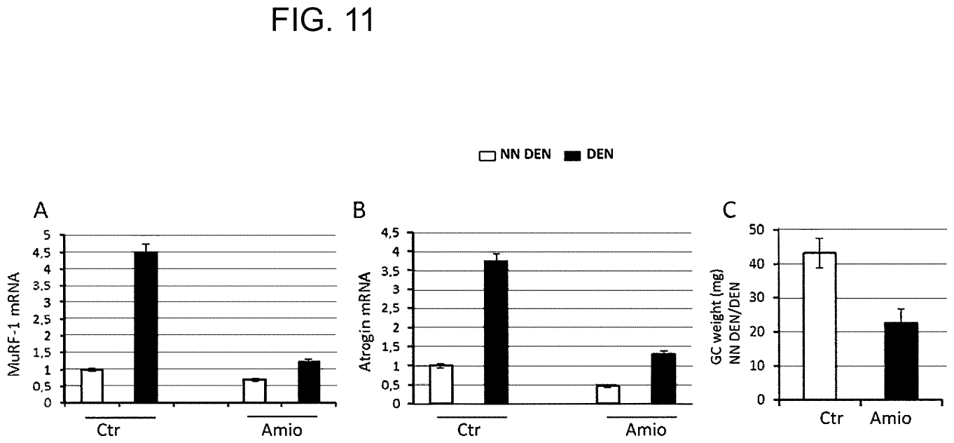

FIG. 11 shows that amiodarone protects against muscle wasting in denervation mice models. (A-B) Graphs showing relative mRNA expression levels of (A) MURF-1 and (B) Atrogin-1 in Ctr (contralateral gastrocnemius muscle) and DEN (denervated gastrocnemius muscle following transection of the sciatic nerve in mouse), with or without amiodarone treatment. (C) Graph showing muscle mass expressed in mg as the difference between gastrocnemius muscle Ctr and gastrocnemius muscle DEN (ΔGC WEIGHT, NN DEN/DEN);

FIG. 12 shows graphs (A, B) illustrating the time-course of the climbing index of dystrophic D. melanogaster flies administered with amiodarone at increasing concentrations, as compared with no treatment and wild-type flies. Results are expressed as the percentage of flies that climbed up to the 15 cm mark of the vial after 60 seconds. To facilitate data visualization, in panel (B) only data obtained following flies administration with 10 μM amiodarone are shown. (C) graph illustrating the day of treatment at which the 50% of dystrophic D. melanogaster population lost its climbing ability following treatment with amiodarone at increasing concentrations, as compared with no treatment and wild-type flies. Comparison of fits: parameters of second order polynomial (quadratic) curves are different for each data set (p<0.0001, extra sum-of-squares F test);

FIG. 13 illustrates the climbing ability of dystrophic D. melanogaster flies following 10 days (A), 15 days (B), 20 days (C) of treatment with amiodarone at increasing concentrations, as compared with no treatment and wild-type flies. In graph (D) it is provided a synoptic view of the above data. Results are expressed as the percentage of flies that climbed up to the 15 cm mark of the vial after 60 sec. * p<0.01 and ** p<0.001 vs the respective Dys+DMSO.

### MATERIALS AND METHODS

### Animals

Animals were house and maintained in the animal facility at CEINGE Biotecnologie Avanzate, Naples, Italy. Experiments and animal care were performed in accordance with institutional guidelines. Tg: Pax7-nGFP; Pax7CreER were provided by Shahragim Tajbakhsh (Pasteur Institute, Paris, France) (Rocheteau, 2012 Cell 148(1-2): 112-125), Dio2 flox/flox (Luongo, 2015 Endocrinology 156(2): 745-754), D2-3xFLAG (Castagna, 2017 J Clin Endocrinol Metab 102(5): 1623-1630), global-D2KO (Christoffolete, 2007 Endocrinology 148(3): 954-960) were used in this study. C57BL/6 and Balb/c were purchased from Jackson Laboratory (Stock No: 000664 and 000651). All mice used for experiments were adults, between 12-16 weeks of age. Both sexes were used for experiments as indicated. Animals were genotyped by PCR using tail DNA.

### Animal Study Approval

All animal studies were conducted in accordance with the guidelines of the Ministero della Salute and were approved by the Institutional Animal Care and Use Committee (IACUC: 167/2015-PR and 354/2019-PR).

### Cell Cultures, Transfections, and Reagents

Primary muscle cultures (pp6) were isolated as described (Qu et al., 1998 J Cell Biol 142(5): 1257-1267) from the indicated mouse lines. Immortalized myoblasts (C2C12) were obtained from ATCC. Proliferating cells were cultured in 20% Fetal Bovine Serum Dulbecco's modified Eagle's medium at 37° C. with penicillin/streptomycin. Transient transfections were performed using Lipofectamine 2000 (Life Technologies) according to the manufacturer's instructions. Anti-MyoD (sc-304) and tubulin (sc-8035) antibodies were purchased from Santa Cruz Biotechnology. Anti-Pax7 antibody were from Developmental Studies Hybridoma Bank. Anti-PARP was from Cell Signaling Technology.

### Muscle Injury

Animals were anesthetized by a ketaminexylazine cocktail, and 12,5 μl of 60 μg/ml CTX (Naja mossambica mossambica, Sigma-Aldrich) was injected into and along the length of the right tibialis anterior (TA) (Yan, 2003 J Biol Chem 278(10): 8826-8836) and 25 μl into gastrocnemius muscle. CTX was injected on different day from Tam.

### Cross Sectional Area (CSA)

The tibialis anterior (TA) muscles were dissected and fixed in isopentane in liquid nitrogen, and sliced into 7 μm sections. The cross-sectional area was analyzed and quantified using CellF*Olympus Imaging Software.

### Immunofluorescence and Histology

Dissected muscles were snap frozen in liquid nitrogen-cooled isopentane, sectioned (7 μm thick) and stained. For immunofluorescent staining, cells or section were fixed with 4% formaldehyde (PFA) and permeabilized in 0.1% Triton X-100, then blocked with 0.5% goat serum and incubated with primary antibody. Alexa Fluor™ 594/647-conjugated secondary antibody was used. Images were acquired with an IX51 Olympus microscope and the Cell*F software. For hematoxylin/eosin staining (H&E), sections were fixed for 15 min and placed in hematoxylin for 5 min followed washed, and 5 min in Eosin.

### Wound Preparation, Macroscopic Examination and Histological Analyses

The back fur of mice was shaved and the skin was cleaned with 70% alcohol. The dorsal skin was pulled using forceps and two 8-mm full-thickness skin wounds were created along the midline using a sterile 8 mm circular biopsy punch by pressing through both layers of the skin pull. Skin wound healing was measured every 2-3 days by anesthetizing the animals and imaging the wounded area.

Excisional, full-thickness skin wounds were aseptically made on the dorsal skin by picking up a fold skin at the midline and using a sterile, disposable biopsy punch with a diameter of 8 mm to punch through the two layers of skin. In this manner, two wounds were made on each side of the midline at the same time. Each wound site was digitally photographed using the Nikon FX-35A at the indicated time intervals, and wound areas were determined on photographs using CellF*. Changes in wound areas were expressed as the percentage of the initial wound areas.

gPCR Experiments

Total RNA was extracted from sorted or cultured cells with a Qiagen RNeasy Micro kit according to the manufacturer's instructions (Qiagen, Hilden, Germany) and then reverse-transcribed into cDNA by using VILO reverse transcriptase (Invitrogen Life technologies Ltd). For real-time PCR, SYBR Green Master mix (Biorad) was used. Cyclofilin A was used as the endogenous control to normalize expression.

### Western Blot Analysis

Total protein extracts from cells and tissues were run on a 10% sodium dodecyl sulfate-polyacrylamide gel electrophoresis gel and transferred to an Immobilon-P transfer membrane (Millipore, Billerica, MA). The membrane was then blocked with 5% nonfat dry milk in phosphate buffered saline, probed with the primary antibody, washed and incubated with horseradish peroxidase-conjugated donkey anti-rabbit or mouse immunoglobulin G secondary antibody (1:3000), and detected by chemiluminescence (Millipore). After extensive washing, the membrane was incubated with antitubulin-specific or ERK antibody (1:10,000; sc-8035, Santa Cruz, Dallas, TX) as loading control.

### Muscle Wasting Mouse Model

### Denervation Experiment

Animals were anesthetized by a ketaminexylazine cocktail and a small incision (≤0.5 mm) was made in the left tight skin. The sciatic nerve was exposed with the help of a small surgical hook and then cut before the separation of sciatic nerve branches. A small (2-5 mm) nerve section was removed to prevent re-innervation. Denervation was performed unilaterally, using the contralateral hindlimb as a negative control. Tissues were collected 2, 6, 12 or 15 days after denervation surgery.

In denervation+/−Amiodarone experiments, mice were treated for 13 days with Amiodarone and were sacrificed 5 days post induced-denervation (from day −7 up day+5 upon denervation).

### Muscle Wasting Mouse Model

### Cachexia Mouse Model

Murine colon 26 adenocarcinoma cells (C26) were cultured in DMEM containing 10% fetal bovine serum, 100 U/ml penicillin, and 100 μg/ml streptomycin. For in vivo inoculation, a single suspension of 7×10⁶ cells in 100 μl of physiological saline was injected subcutaneously into the right flank of the BALB/c mice. The same volume of physiological saline was injected into the control groups.

In cachexia+/−Amiodarone experiments, mice were treated for 18 days with Amiodarone and were sacrificed 7 days post induced-cachexia. Amiodarone was given from day-7 to day +7 (day 1 is the day of cells injection).

### Muscle Wasting Mouse Model

### Fasting Experiment

C57BL6 mice were fasted for 6, 12 and 24 hours with unrestricted water access.

### rT3 Administration

In vivo: mice (C56BL6 mice) were treated systemically with an i.v. injection of 100 μl of 30 ng/μl rT3 (Sigma Aldrich) or PBS vehicle control to consecutive 4 days.

In vitro: Pp6 or C2C12 cells were treated with rT3 (30 nM)

### Amiodarone Administration

Mice (C57BL6 mice) were treated with 0.45 mg/ml Amiodarone (Sigma, A8423, St. Louis, MO, USA) (Bagchiet al., Circulation Research 1987; 60:621-625; Lee et al., Oncotarget, 2015; 6(40): 42976-42987) in drinking water ad libitum starting 1 week before the induced-denervation or cachexia, until sacrifice.

Preparation of amiodarone: 45 mg dissolved in 1 ml of water for 10 minutes at 80° C.

### TNFα Treatment

Pp6 or C2C12 cells were treated with TNFα (40 ng/μl) for 24 hours.

Drosophila melanogaster Model of Muscular Dystrophy

### Drosophila Strain

The genetic loss-of-function homozygous Drosophila mutant for dystrophin (dys), DysE17 (hiips://flybase.org/reports/FBal0241311.html) was employed as a Duchenne muscular dystrophy model, which carries a point mutation on chromosome 3 (location: 92A10, 3R:19,590,458 . . . 19,590,458) causing a nucleotide change C19590458T and consequently the amino acid change Q2807term|Dys-PA. This is a termination codon determining full-length dys protein to be abnormally shortened. As control, the wild-type Oregon-R strain was used.

The adult wild-type animals and mutant fruit flies were treated with amiodarone at increasing concentrations, supplemented in the food for 30 days.

Husbandry: flies were raised on a standard corn meal agar food (pH 5.5) at 25° C. with minor modifications. Fly food was prepared as follows: 100 g of yellow cornmeal, 100 g of brewer's yeast, 8 g of agar and 75 g of sucrose (5% w/v) were mixed and dissolved by adding warm plain water to a final volume of 1.5 l, the hydration source of the flies. The mixture was autoclaved and allowed to cool down slowly. The broad spectrum fungicide Nipagin (3 g dissolved in 16 ml of absolute ethanol) was added when the temperature reached approximately 50° C., and the mixture was then dispensed into vials.

Mating: populations of young adult flies (3 days old) were placed in vials for mating and eggs laying. Vials were visually inspected to ensure copulation was occurring; within 5-30 minutes, all females were typically paired with males. Individual eggs were gently picked after a 24 h copulation of untreated adults using 2% agar plates supplemented with apple juice. Eggs were then washed in PBS, counted and separated under a stereomicroscope. At around day 5 from mating, third instar larvae emerged from food were sampled. To note, the culture population of dystrophic Drosophila is a mix of heterozygous and homozygous mutants for DysE17. In heterozygous individuals, the third chromosome alleles are balanced with the TM6, Tb balancer chromosome. The balancer allows distinguishing the larvae (heterozygous vs homozygous) on basis of reduced length and tortuous tracheal trunks. The expected proportion of homozygous DysE17 progeny is around 30% of total.

### Amiodarone Administration

The amiodarone compound was dissolved in DMSO as a stock solution and aliquoted at −20° C.

Eclosed Oregon-R and homozygous DysE17 Drosophila (1-2 days of adult age) were placed in vials (10-15 animals/vials) and reared on 4-5 ml of either the standard medium or medium supplemented with amiodarone at increasing doses (100 nM, 1 μM, 10 μM, 20 μM, and 50 μM). Food was replaced each 5 days. Amiodarone-supplemented diets were freshly prepared by mixing amiodarone with appropriate amounts of standard food just before the administration to animals. At least 3 vials per group were prepared within each experiment. Each experiment was repeated 3 or more times: in total, a minimum of 60 animals per experimental condition was analysed.

### Groups of Treatment:

Oregon-R receiving vehicle (DMSO 1:1000);
DysE17 flies receiving vehicle (DMSO 1:1000);
DysE17 flies receiving 100 nM amiodarone;
DysE17 flies receiving 1 μM amiodarone;
DysE17 flies receiving 10 μM amiodarone;
DysE17 flies receiving 20 μM amiodarone;
DysE17 flies receiving 50 μM amiodarone.

Flies were treated for 30 days (treatments ended when animals reached 30 days of adult age), with food renewal each 5 days.

### Climbing Assay

Geotaxis was assessed using a climbing assay (negative geotaxis reflex in opposition to the Earth's gravity). A horizontal line was drawn 15 cm above the bottom of the vial. After a 10 minutes rest period, the flies were tapped to the bottom of the vials: all flies were forced to start climbing (vertical walking). The number of flies that climbed up to the 15 cm mark after 60 seconds was recorded as the percentage success rate. A camera recorded fly movement during the experiment. Each trial was performed three times, at one-minute intervals, and the results averaged. Climbing performance was assessed each 5 days starting from day 1-2 of adult age (just after eclosion) and ending at 30 days.

### Statistical Analysis

Significant differences were determined using one-way ANOVA, Mann-Whitney and the Student's t test, with p<0.05 considered as statically significant. All statistics and graphics were performed using GraphPad Prism7 and appear in more detail in SI Appendix, S20. In all figures, error bars represent the SEM. A value of p<0.05 was recognized as significant (*P<0.05; ** P<0.01; *** P<0.001).

### Results

### Example 1: D2 is Expressed in Quiescent Stem Cells of Muscle and Skin

The present inventors analyzed D2 expression by immunofluorescence in the previously characterized 3xFLAG-D2 knock-in mouse (Castagna, 2017 J Clin Endocrinol Metab 102(5): 1623-1630). With their experiments, the inventors found that D2 co-localized with the muscle stem cell marker Pax7+(Seale, 2000 Cell 102(6): 777-786) in resting tibialis anterior (TA) muscle (FIG. 1A). Interestingly, D2 expression co-localized with the hair follicle stem-cell marker CD34, which specifically marks the bulge of the hair follicle, where quiescent stem cells are localized (FIG. 1B).

Overall, these findings indicate that D2 mark quiescent stem cells.

### Example 2: Type 2 Deiodinase is Induced in Muscle in Different Models of Muscle Wasting

Moreover, the inventors analyzed TH metabolism in skeletal muscle atrophy by means of three different models of muscle atrophy, i.e. denervation, cancer cachexia and fasting. Denervation was induced in two-months old mice by sciatic nerve rescission of one leg. The contralateral leg was used as internal control. In denervated mice, collected at different time points following denervation, mRNA levels of different TH modulators (D2, D3, TRα, TRß, MCT8 and MCT10) were measured. D2 mRNA and protein was sharply up-regulated six days following denervation and its expression was sustained till day 15 (FIG. 2A, D). Analogously, D2 expression was enhanced in cachexia, in BalbC mice injected subcutaneously (s.c.) with 7×10⁶ colon 26 adenocarcinoma cells (C26) (FIG. 2B). Also fasting induced a similar increase in D2 expression in the early time of starvation (FIG. 2C). These results suggest that induction of D2 and, consequently, a local amplification of TH-signaling, is a common feature of different models of muscle atrophy.

### Example 3: D2 Depletion Enhances Stem Cell Proliferation During Muscle Regeneration

To determine the consequences of D2 depletion in satellite cells upon injury, the inventors analyzed TA muscles from satellite-cell specific cD2KO mice collected 21 days after Cardiotoxin (CTX) injection (FIG. 3A).

D2-depletion resulted in a greater number of centrally nucleated fibers versus control, associated with a smaller fiber diameter (FIGS. 3B-C). Accordingly, the number of Pax7+/MyoD+ cells, and the equivalent Pax7+/BrdU+ cells increased while the number of Pax7+/MyoD− and the Pax7−/MyoD+ cell population decreased (FIG. 3D). This suggested that the absence of D2 increases the proliferative capacity of stem cells while the completion of myogenic program is delayed.

Interestingly, 60 days after CTX injury, fiber diameter was larger in cD2KO mice versus control, which suggests that complete maturation can be achieved with prolonged time in the absence of D2 (FIG. 3E).

### Example 4: D2 Depletion Enhances Stem Cell Proliferation During Skin Regeneration

To characterize the effects of D2-depletion in vivo during tissue regeneration, wound healing assays were performed by the inventors. Time course of regenerating epidermis showed that the closure of the wound was significantly faster in D2-depleted epidermis than control skin (FIG. 4A). Overall, these data point to a key role of D2 in preserving hair follicle stem cells from uncontrolled activation.

### Example 5: global D2 depletion in mice attenuates muscle wasting

To assess the functional role of D2 induction in muscle atrophy, the inventors performed cancer cachexia studies in D2KO mice. Matched pairs of D2KO and wild-type (WT) mice were injected with C26 cells and muscle wasting was monitored at 12 and 15 days following injection.

WT muscles underwent dramatic muscle wasting from day 8-9 till day 15 following injection as demonstrated by Hematoxylin and Eosin staining, fiber cross sectional area (CSA), body and heart weight (FIGS. 5A-D). Conversely, D2KO muscles were preserved from massive muscle wasting and till 15 days following injection, the atrophic process was attenuated compared to WT mice (FIGS. 5A-D).

Importantly, while the mean viability of WT mice was 12.7 days, D2KO mice almost doubled their viability (22.4 days, FIG. 5D). Interestingly, atrogenes induction, a common sign of ongoing muscle atrophy, was severely reduced in D2KO mice (FIGS. 5E and F).

Muscle denervation studies in D2KO mice showed a similar phenotype of reduced muscle wasting in D2KO mice compared to WT mice. At 9 and 12 days after denervation, gastrocnemius (GC) weight of WT animals was reduced by 38% and 34%, respectively, while the loss of muscle weight in D2KO mice was of 4% and 6% (FIG. 6A). Consistently, diameter of D2KO mice fibers was remarkably higher at 12 days after denervation, when compared to WT mice (FIG. 6B).

The ubiquitin-ligases MuRF-1 and Atrogin-1 were also potently down-regulated in denervated D2KO mice compared to denervated WT mice, indicating that the UPS stimulation is drastically reduced in the absence of D2 (FIGS. 6C and D).

These data prove the efficiency of D2 ablation and the consequent reduction in TH action for drastic attenuation of experimentally induced atrophy in vivo.

### Example 6: Drug-Induced D2-Inhibition as Novel Therapeutic Tool to Activate Stem Cells

To therapeutically exploit the capacity of D2-depletion to activate stem cells and accelerate muscle tissue repair, mice were treated with oral reverse triiodothyronine (rT3, a specific D2-inhibitor) for 7 days (−5/+2 relative to CTX injury) (FIG. 7A). Hematoxylin & Eosin analysis 7 days post-CTX showed the presence of an increased number of fibers, that were also smaller than control in rT3 treated mice (FIGS. 7B and 7C). In rT3 treated mice, Pax7+/EdU+ cells were increased than control (FIG. 7D). Importantly, 21 days after injury larger fibers were observed in rT3 treated mice than control, in agreement with the limited time frame (7 days) of D2 inhibition (FIGS. 7E and 7F).

In a similar setting, the present inventors also tested whether D2 inhibition positively affected wound regeneration in skin. Indeed, rT3-treated mice repair wounds more rapidly and efficiently than control (FIGS. 8A-B). Interestingly, at later time points following skin wounding, it was observed that animals treated with rT3 had more complete regrowth of hair in the shaved area (FIG. 8D).

Taken together, these results demonstrated that D2-blocking with rT3 by “activating” stem cells facilitate regeneration in different tissue contexts and could be therapeutically exploited in vivo.

### Example 7: TH Regulates NFKB/TNFα Signaling Pathway

During muscle atrophy most proinflammatory cytokines such as tumor necrosis factor (TNF)-alpha, interleukin (IL)-1beta and IL-6 are overexpressed along with enhanced Nuclear Factor Kappa B (NFKB) activation (Bonaldo P. and Sandri M., Dis Model Mech. 2013 January; 6(1):25-39).

The inventors tested the effects of TH modulation upon TNFα treatment of muscle cells. When cells were treated with TNFα in normal serum, cell death was observed at 24h (FIGS. 9A-G). Conversely, cells cultured in the absence of TH (or when TH activation was prevented via D2 blocking by rT3) were protected from apoptosis, while TNFα receptor additional rescued the anti-apoptotic effect of D2-blocking (FIGS. 2A and B). TNF is one of the most potent physiological inducers of the nuclear transcription factor NF-kappa B which mediates many of its biological effects such as apoptosis; here it was demonstrated that NF-KB activation and the consequent p65 subunit translocation to nucleus were blocked by rT3 (FIGS. 9C and D).

The inventors observed that while not changing the total p65 levels, TNFα induced Caspase-8 activity in normal serum grown cells, and this effect was almost absent in the absence of THs (or when TH activation was prevented via D2 blocking by rT3, or when cells were cultured in charcoal stripped medium) (FIG. 9E). In agreement with this, over-expression of D3 in muscle cells protected them from TNF induced apoptosis (FIGS. 9F and G), while the addition of TNF-receptor rescued protective effect of D3 overexpression (FIG. 9G)

Taken together, these data point to a major role for TH in inducing critical mediator of muscle atrophy and confirm the protecting role of D2 inactivation against atrophy-induced muscle cells death.

### Example 8: Drug-Induced D2-Inhibition as Novel Therapeutic Tool to Attenuate Muscle Wasting

To therapeutically exploit the capacity of D2-blocking to protect mice from massive muscle wasting, the present inventors used a specific D2 inhibitor, amiodarone. In tumor-induced cachexia mice models, mice were treated with oral amiodarone for 18 days (−7/+11 relative to C26 injection) (FIG. 10A). Matched group of amiodarone-treated and control mice were injected with C26 cells, and muscle wasting was monitored to different days following injection. Muscles of control mice underwent dramatic muscle wasting as demonstrated by fiber cross sectional area (CSA), body, heart, TA (tibialis anterior) and GC (gastrocnemius muscle) weight (FIGS. 10B-E and 10J). Conversely, muscles of amiodarone-treated mice were preserved from massive muscle wasting (FIG. 10B-E). Furthermore, there were no significant effects on tumor weight between the amiodarone-treated and control mice, indicating that the treatment with amiodarone did not inhibit tumor growth (FIG. 10F).

The ubiquitin-ligases MuRF-1 and Atrogin-1 were also potently down-regulated in amiodarone-treated mice compared to control (FIGS. 10 G and H). Importantly, while the survival percentage of control mice was about 12.7 days, amiodarone-treated mice almost doubled their survival (more of 27 days, FIG. 10I), similarly to what observed in tumor-induced cachexia in genetic D2-depleted mice.

In denervation mice models, mice were treated with oral amiodarone for 13 days (from day −7 up day+5 upon denervation). In agreement with the tumor-induced cachexia model in D2KO mice, the ubiquitin-ligases MuRF-1 and Atrogin-1 were also potently down-regulated in amiodarone-treated mice compared to control mice (FIGS. 11A and B). Furthermore, also after denervation the muscles of amiodarone-treated mice were preserved from massive muscle wasting as demonstrated by the maintenance of gastrocnemius muscle mass (FIG. 11 C).

These data prove the efficacy of D2 ablation in drastic attenuating experimentally induced muscle atrophy in vivo.

### Example 9: Drug-Induced D2-Inhibition as a Novel Therapeutic Tool for the Functional Recovery of Skeletal Muscle

To further validate the therapeutic potential of D2 inhibitor compounds, the present inventors investigated the effects of amiodarone on skeletal muscle in a fly model characterized by a dystrophic phenotype (Dys^{E17}). Drosophila dystrophyn gene is as complex as its mammalian counterparts since encodes three large-isoforms of dystrophin-like protein (DLP1, DLP2, and DLP3) and three truncated products (Dp186, Dp205 and Dp117).

Complex issues of system function/dysfunction can be investigated in the fruit fly Drosophila melanogaster more rapidly than in other model organisms. Drosophila has the significant strength of allowing specific expression experiments in the context of a powerful and well-established genetic framework. In this respect, representative and highly relevant Drosophila systems of human disease have a key role in drug discovery via target-identification, in vivo repurposing and high-throughput compound screenings, thus favoring the translation of lead compounds into human therapeutics. In the fly life cycle, fly larvae hatch from laid fertilized eggs and eat continuously, stopping only to molt twice after first instar and second instar stages. At 5/6 days after egg laying, third instar larvae leave the food and “wander” as they prepare to undergo metamorphosis into the adult fly (9/10 days). For these reasons D. melanogaster is a suitable organism for in vivo chronic drug delivery from fertilized eggs to adult stages. Also, the feeding experiments in Drosophila are important to support the efficient activity and oral delivery of the molecule in-vivo, as a step towards evaluating the biological effects in a multicellular organism.

For the validation study, the climbing efficiency and muscle structure of adult wild-type animals and DysE17 mutants were analyzed after food supplementation with amiodarone at increasing concentrations for 0-30 days (geotaxis assay). The rate of climbing decay in dystrophic Drosophila mutants is significantly faster than in wild-type flies, suggesting that dystrophin-like proteins are required in the normal musculature. Accordingly, using different stocks, it has been previously demonstrated that Drosophila dys mutants begin adult life with normal mobility opposed to gravity, before showing a time-dependent muscle defects comparable with our model.

As shown in FIG. 12, the climbing ability of DysE17 flies treated for 30 days with 1:1000 DMSO only (the vehicle used for the concentration of 50 M amiodarone) was lower than Oregon wild-type flies. Notably, animal administration with increasing doses of amiodarone from 100 nM to 10 μM induced a dose-dependent beneficial effect on fly mobility with the most effective concentration at 10 μM. Higher concentrations of amiodarone, i.e. 20-50 M, did not alter the climbing decay of dystrophic animals.

The determination of the day of treatment at which only 50% of flies were able to climb (FIG. 12C) allowed to point out further amiodarone effects. Climbing decays: Oregon-24.5 days; Dys-14.2 days; Dys+amiodarone 100 nM-15.5 days; Dys+amiodarone 1 μM-16.5 days; Dys+amiodarone 10 μM-17.5 days; Dys+amiodarone 20 μM-12.8 days; Dys+amiodarone 50 μM-12.3 days.

The analysis of the climbing ability of Dys flies at different days of treatment (FIG. 13) showed that amiodarone-induced increase of mobility was evident (and followed a dose-dependent trend) after 15 and 20 days of treatment while no effects were observed at shorter times. In particular, the climbing decay of dystrophic flies was almost halved following animal treatment with amiodarone at 10 μM for 15 days (35% vs 18% damaged animals) and 20 days (55% vs 32% damaged animals). Notably, after 25-30 days, both untreated and amiodarone-treated Dys animals displayed the same functional deficits (see results above). These results suggest that amiodarone may ameliorate the climbing performance of dystrophic flies within a specific temporal window.

The above-illustrated results of the experimental plan indicate that chronic administration (30 days) of adult dystrophic flies with amiodarone at 10 μM ameliorated significantly the animal climbing efficiency. It is also worth noting that a dose-dependent beneficial trend was observed in the animals using amiodarone at a concentration lower than 10 UM while amiodarone concentrations in the range of 20-50 μM were devoid of effects, suggesting a somewhat detrimental threshold. The highest beneficial effects of amiodarone were achieved after the early-young age of Drosophila, i.e. from 15 to 20 days of treatment, and disappeared in the middle aged-old flies (after 25 days). The functionally recovered muscles of the dystrophic mutant strains of D. melanogaster were still compromised at structural level.

Taken together these results show that amiodarone positively affects skeletal muscle contractility but not the disorganization of dystrophic myofibrils.

1-10. (canceled)

11. A method for the therapeutic treatment of muscle wasting and/or muscle disease and/or skin disease, comprising administering to a subject in need thereof an effective amount of an inhibitor compound of type 2 iodothyronine deiodinase (D2).

12. The method according to claim 11, wherein said inhibitor compound of type 2 iodothyronine deiodinase (D2) is selected from the group consisting of reverse triiodothyronine (rT3), amiodarone (AMIO), dese-thylamiodarone (DEA), 5-methyl-2-thiouracil (MTU), 6-benzyl-2-thiouracil (BTU), xanthohumol (XTH), genistein (GEN), 6-propyl-2-thiouracil (PTU), methimazole (MMI), iopanoic acid (IAc), dexamethasone, gold thioglucose (GTG), and any combination thereof.

13. The method according to claim 11, wherein said muscle wasting is due to a disease condition selected from the group consisting of sarcopenia, cancer, sepsis, diabetes, chronic heart failure, chronic obstructive pulmonary disease, chronic renal failure, liver cirrhosis, cystic fibrosis, muscle denervation, fasting, and any combination thereof.

14. The method according to claim 13, wherein said disease condition is neoplastic cachexia.

15. The method according to claim 11, which is for the therapeutic treatment of a muscle disease selected from the group consisting of delayed-onset muscle soreness (DOMS), muscular contusion, muscular strain, muscular laceration, cancer cachexia, muscle atrophy, muscle dystrophy, and any combination thereof.

16. The method according to claim 11, which is for the therapeutic treatment of a skin disease selected from the group consisting of ulcers, dermatitis, skin wounds, burns, lacerations, and any combination thereof.

17. The method according to claim 12, which is for the therapeutic treatment of a muscle disease selected from the group consisting of delayed-onset muscle soreness (DOMS), muscular contusion, muscular strain, muscular laceration, cancer cachexia, muscle atrophy, muscle dystrophy, and any combination thereof.

18. The method according to claim 12, which is for the therapeutic treatment of a skin disease selected from the group consisting of ulcers, dermatitis, skin wounds, burns, lacerations, and any combination thereof.

19. The method according to claim 12, wherein said muscle wasting is due to a disease condition selected from the group consisting of sarcopenia, cancer, sepsis, diabetes, chronic heart failure, chronic obstructive pulmonary disease, chronic renal failure, liver cirrhosis, cystic fibrosis, muscle denervation, fasting, and any combination thereof.

20. A method for the therapeutic treatment of muscle wasting and/or muscle disease and/or skin disease, comprising administering to a subject in need thereof a pharmaceutical composition comprising a therapeutically effective amount of at least one inhibitor compound of type 2 iodothyronine deiodinase (D2) and a pharmaceutically acceptable vehicle, excipient and/or diluent.

21. The method according to claim 20, wherein said inhibitor compound of type 2 iodothyronine deiodinase (D2) is selected from the group consisting of reverse triiodothyronine (rT3), amiodarone (AMIO), dese-thylamiodarone (DEA), 5-methyl-2-thiouracil (MTU), 6-benzyl-2-thiouracil (BTU), xanthohumol (XTH), genistein (GEN), 6-propyl-2-thiouracil (PTU), methimazole (MMI), iopanoic acid (IAc), dexamethasone, gold thioglucose (GTG), and any combination thereof.

22. The method according to claim 20, wherein said muscle wasting is due to a disease condition selected from the group consisting of sarcopenia, cancer, sepsis, diabetes, chronic heart failure, chronic obstructive pulmonary disease, chronic renal failure, liver cirrhosis, cystic fibrosis, muscle denervation, fasting, and any combination thereof.

23. The method according to claim 22, wherein said disease condition is neoplastic cachexia.

24. The method according to claim 20, which is for the therapeutic treatment of a muscle disease selected from the group consisting of delayed-onset muscle soreness (DOMS), muscular contusion, muscular strain, muscular laceration, cancer cachexia, muscle atrophy, muscle dystrophy, and any combination thereof.

25. The method according to claim 20, which is for the therapeutic treatment of a skin disease selected from the group consisting of ulcers, dermatitis, skin wounds, burns, lacerations, and any combination thereof.

26. The method according to claim 21, which is for the therapeutic treatment of a muscle disease selected from the group consisting of delayed-onset muscle soreness (DOMS), muscular contusion, muscular strain, muscular laceration, cancer cachexia, muscle atrophy, muscle dystrophy, and any combination thereof.

27. The method according to claim 21, which is for the therapeutic treatment of a skin disease selected from the group consisting of ulcers, dermatitis, skin wounds, burns, lacerations, and any combination thereof.

28. The method according to claim 21, wherein said muscle wasting is due to a disease condition selected from the group consisting of sarcopenia, cancer, sepsis, diabetes, chronic heart failure, chronic obstructive pulmonary disease, chronic renal failure, liver cirrhosis, cystic fibrosis, muscle denervation, fasting, and any combination thereof.
